(19) United States
(12) Patent Application Publication
(10) Pub. No.: US 2026/0137490 A1
(43) Pub. Date: May 21, 2026

(54) SYSTEM FOR DETECTING AND TRACKING SCAN BODIES WITH OPTICALLY DETECTABLE FEATURES

(71) Applicant: X-Nav Technologies LLC, Lansdale, PA (US)

(72) Inventors: Scott Merritt, Golden, CO (US); Edward Marandola, Gwynedd, PA (US)

(21) Appl. No.: 19/441,515

(22) Filed: Jan. 6, 2026

Related U.S. Application Data
(63) Continuation of application No. 18/176,109, filed on Feb. 28, 2023.

(51) Int. Cl. A61C 9/00; A61B 90/00; G06T 7/00

(52) U.S. Cl. CPC A61C 9/0053; A61B 90/39; G06T 7/0014; A61B 2090/3937

(57) ABSTRACT

An optical imaging system for detecting and locating an implant fixture in the oral cavity of a patient using one or more scan bodies. The system includes two or more spaced apart cameras for capturing images of a common location. At least one memory including stored data representing (i) a reference dataset of optically detectable characteristics from one or more scan bodies, and (ii) a three dimension model of the patient's oral cavity from a prior scan of the patient's oral cavity. At least one processor is programmed to (a) receive image data from the cameras representing the common location, (b) analyze the image data to locate optically detectable characteristics associated with one or more scan bodies, (c) compare the image data to the reference dataset, (d) determine the pose and location of an implant in the image data based on the comparison, and send data to a display.

### RELATED APPLICATION

This application is a continuation of U.S. patent application Ser. No. 18/176,109, filed Feb. 28, 2023, which is related to and claims priority from U.S. Provisional Patent Application 63/316,509, filed Mar. 4, 2022, the disclosures of which are both incorporated herein by reference in their entireties.

### FIELD OF THE INVENTION

The present invention is directed to use of scan bodies in image guided surgery and, more particularly, to the use of scan bodies that include optically detectable features, such as physical characteristics or laser etches indicia, during an optical imaging procedure, such as part of image guided oral surgery, for detecting and locating the scan bodies.

### BACKGROUND

To replace a broken or damaged tooth a patient will typically undergo a surgical procedure in which a dentist drills a hole into the jawbone in the location of the missing or removed tooth/teeth, and inserts and secures a metal implant fixture. The implant includes a cavity into which an abutment and, subsequently, a dental prosthesis is later attached. Previously it was common for a dentist to make an impression of the implant and abutment. Once the impression material hardened, it was removed and then sent to a lab to create a dental mold, in stone form, of the patient's teeth with the abutment representing the position of the implant in the patient's mouth.

More recently, devices called “scan bodies” have been used to permit digital imaging of the implant location, scan bodies, and surrounding teeth. The scan bodies consist of three dimensional shaped bodies or posts that are removably attached to the implant fixture. They extend above the surrounding tissue and represent the position and orientation of the implant fixture. After attachment, the scan body and surrounding tissue and teeth are scanned using an inter-oral scanner to create a digital representation of the scan body and surrounding features. The digital scanned image allows the lab and the restorative dentist to create the final abutment and crown based on the position and orientation of the scan body.

### BRIEF DESCRIPTION OF THE DRAWINGS

For the purpose of illustrating the invention, the drawings show a form of the invention that is presently preferred. However, it should be understood that the invention is not limited to the precise arrangement and instrumentalities show in the drawings.

FIG. 1A is a perspective view of a jawbone of a patient illustrating a mounted implant fixture.

FIG. 1B is a perspective view of the jawbone of FIG. 1A illustrating a mounted scan body according to the present invention.

FIG. 2 is an illustration of a scan body with graphical indicia depicted on it according to one embodiment of the invention.

FIG. 3 is a flat pattern illustration of the outer surface of a scan body illustrating patterns according to one embodiment of the invention.

FIG. 4 is an illustration of an image tracking system for capturing the location of a scan according to one embodiment of the present invention.

FIG. 5 is an illustration of a scan body with optically detectable features according to an embodiment of the invention.

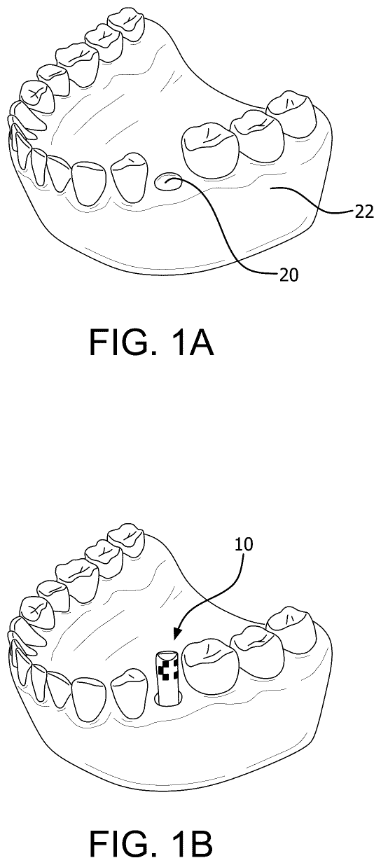

### BRIEF DESCRIPTION OF THE PREFERRED EMBODIMENTS

Referring now to the drawings which illustrate one or more preferred embodiments of the invention, the invention is directed to improved scan bodies 10 and to a method of use of those scan bodies during or as part of image guided surgery. The scan bodies 10 comprise a hollow or substantially hollow body member 12 with an outer surface 14 and an inner cavity 16 extending from the top to the bottom of the body member 12. As is convention in the art, the cavity 16 is configured to receive a fastener 18, such as a screw, for securing the body member 12 to an implant 20 formed in the jawbone 22 of a patient in a conventional manner. The body member 12 is preferably made of material that is safe to place in a patient's mouth. In one embodiment, the body member is made from thermoplastic material, such as PEEK (polyether ether ketone) with a titanium base and screw. Any suitable material that is preferably autoclavable and reusable can be used.

The fastener 18 is inserted through an opening 24 at or near the top of the body member 12 and engages threads in the body member 12 and the implant fixture 20, thereby securing the body member 12 to the implant fixture 20.

In an embodiment, graphical tracking indicia 30 is formed on the outer surface 14 of the body member 12. More specifically, the graphical optically detectable indicia 30 is one or more optically detectable patterns formed or placed on the outer surface of the body member 12. The optically detectable patterns 30 are optically visible patterns that can be detected and captured by externally mounted cameras and from which a computer processor can determine visual reference points that allow the position and location of the scan bodies 12 in the patient's mouth to be determined. From that information, the location of the implant fixture can be calculated by the processor/system.

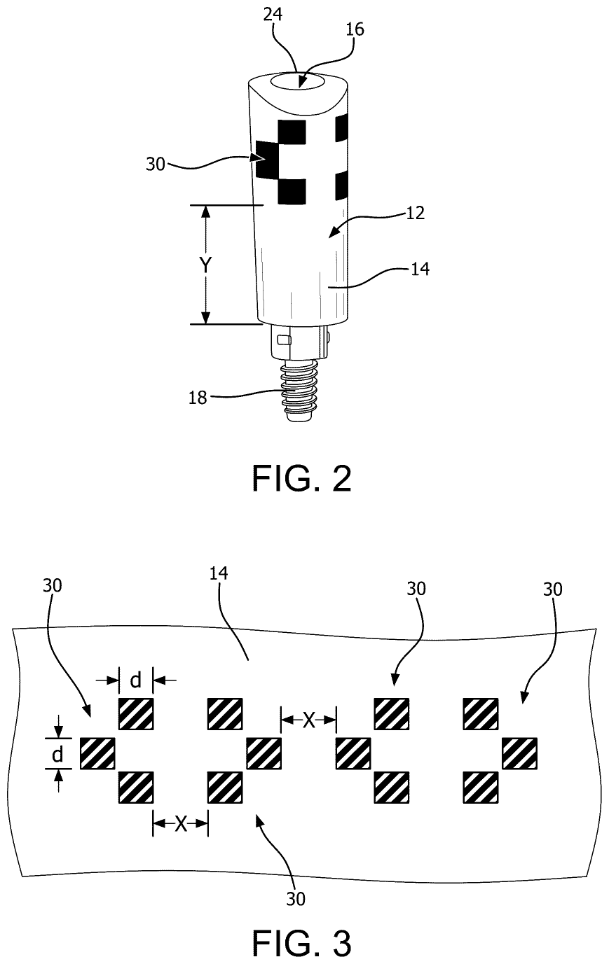

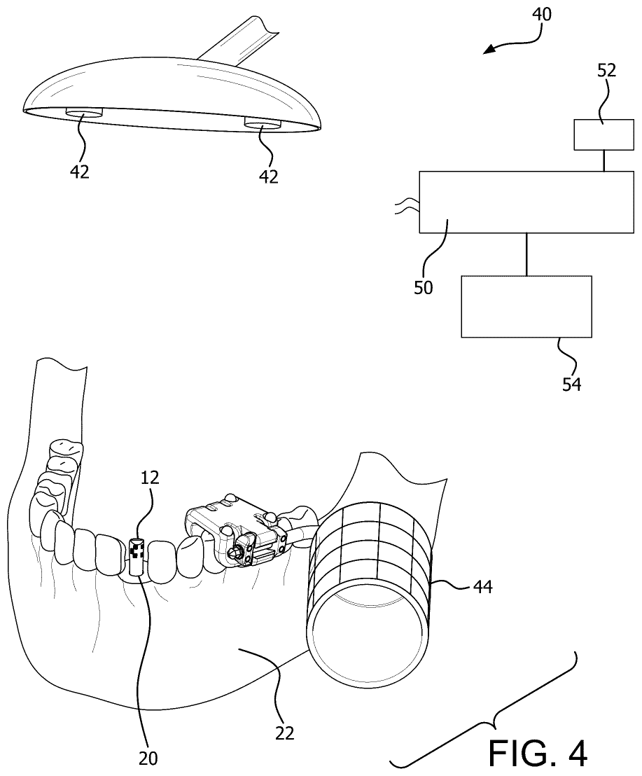

In one embodiment, each tracking pattern 30 comprises a plurality of precisely located images that can be detected and compared to a library or stored records of preset patterns. For example, in the illustrated embodiment shown in FIGS. 2 and 3, there are multiple patterns 30 spaced apart X around the circumference of the outer surface 14 of the scan body, preferably at least three or four patterns. Each pattern comprises at least three squares arranged in a predetermined order. In order to properly determined the rotational orientation of the scan body, adjacent patterns preferably do not repeat themselves. That is, each pattern 30 is different from its neighboring or adjacent patterns. In the illustrated embodiment the difference is a simple mirror imaging of the adjacent pattern, but adjacent patterns can be completely distinct. While the illustrated embodiment shows the pattern consisting of squares, other detectable patterns can be used. U.S. Pat. Nos. 9,402,691 and 9,943,374, incorporated herein by reference in their entireties, describe different types and arrangements of tracking patterns, processes for detecting points in patterns and calculating three dimensional locations and orientations based on the optically detectable patterns. The optically detectable pattern may be molded or etched into (such as high contrast pattern engraving) or disposed or placed on the outer surface of the scan body 12.

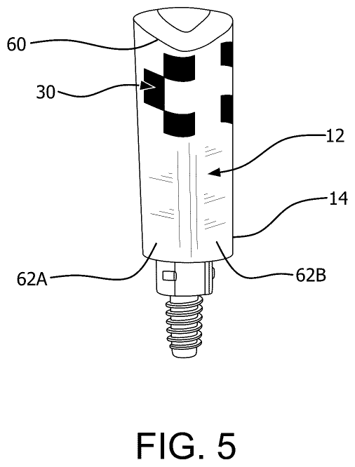

The present invention also contemplates that the optically detectable patterns/indicia may not be the primary mechanism for determining the placement and orientation of the scan bodies. Instead, in an embodiment, the system detects the shapes and/or features of the scan bodies captured in the scanned images to determine their location. More specifically, the processor is programmed to detect characteristics of images of the scan bodies, such as the shape and/or physical, material and/or reflectance characteristics, of the scan bodies, for detecting the scan bodies and determining their location and orientation. See, FIG. 5 which illustrates a scan body 12 with an outer surface 14 that includes physical characteristics, such as facets or edges 60, as well as reflective characteristics, such as surface finish, that result in different reflectance (62_{A} vs. 62_{B}) from the lighting. The processor is programmed to either 1) model the appearance of the object using known or detected lighting conditions and determining the alignment of the object that best describes the observed images; or 2) detect discontinuities of the object in the image and aligning them with facets in a prestored model of the scanned body; or 3) a combination thereof. In this embodiment, the indicia may be used as part of a secondary analysis in the event that the determination of the location and orientation of the scan bodies based on detecting the shape or characteristics/features of the scan body results in any ambiguity. For example, ambiguity might arise in from certain symmetries of the scan body, e.g., the spin orientation of a perfectly cylindrical scan body. In such cases, optical detection of the indicia can be used to resolve the ambiguity.

In an embodiment, the modeling of the appearance of the object is accomplished, for example, by determining the 3D location of the light source relative to the cameras' axes. If there are multiple light sources, assuming that their arrangement/location are relatively close, for simplicity the light sources can be assumed to originate from a point source. However, for more complex light source arrangements it may be more appropriate to determine the 3D location of the different light sources, which a person skilled in the art would be able to implement using known techniques.

The system is programmed to model the reflectance properties of the scan bodies. For example, the model may include properties such as how “shiny” or brightly reflective and/or matted a scan body (or portions of it) are, and the relative contrast between different portions of the scan body. For example, certain faceted edges of a scan body will yield a different reflectance than a smoothly curved or cylindrical portion.

Once the system knows where the object is relative to the camera, the processor is programmed to use 1) stored values representing the camera's intrinsic characteristics (focal length and distortion), 2) the light source(s) location(s) and extent(s), 3) the object's reflectance and albedo (reflective power) properties, and 4) the pose (3D location and orientation) of the object in the scene to create a 3D rendering using conventional computer graphics methods, such as Phong surface rendering/shading (see, for example, “Blinn-Phong reflection model”, Wikipedia, The Free Encyclopedia, incorporated herein by reference in its entirety) or ray-tracing (see, for example, “Ray tracing (graphics)”, Wikipedia, The Free Encyclopedia, incorporated herein by reference in its entirety), which simulate the appearance of the object in each camera. From that information, the program can determine the pose of the object by using an optimization algorithm, which generically is for each of the different poses covering a range of possible poses, such as by (i) rendering the simulated image, (ii) comparing the simulated image with the actual image and computing a visual similarity, for example, normalized cross-correlation, or simple pixel difference, and (iii) identifying the pose that generated the best similarity score.

It is contemplated that different conventional optimization algorithms can be used, such as, grid search, Powell's method, “Powell's method”, Wikipedia, The Free Encyclopedia, incorporated herein by reference in its entirety), or gradient methods, such as gauss-newton and Levenberg Marquardt, “Levenberg-Marquardt algorithm”, Wikipedia, The Free Encyclopedia,, incorporated herein by reference in its entirety).

In an embodiment, discontinuities (contrasting boundaries) in the image are characterized by changes in intensity or color. Thus, discontinuities will result in high gradient magnitudes. Facets in the model are characterized by step changes in the normal directions along edges of the geometry (e.g., two triangles with very different normals sharing a single edge). Assuming a known pose, the set of facet edges can be re-projected into the same coordinate system as the camera image by using common techniques known in computer graphics, and the information determined in items 1, 2, and 4 above. The edges can then be correlated with the gradient magnitudes and directions in the image. For example, if the model is well-aligned with the image, the pixels along the reprojected facet edges should have a higher gradient magnitude than other pixels, and the gradient should be orthogonal (or nearly so) to the re-projected facet edge. Using an optimization algorithm like before, one can search the pose space to find the pose whose edges are best aligned to the facets of the aligned model.

While the above description describes matching of the actual scanned body to a prior model of a scanned body based on detection of edges, discontinuities or facets, it is also possible to detect and model smoothly curved surfaces. For example, the present invention can detect variations in surface reflections of smoothly-varying surface normals across the surface contour. In this embodiment, the system is programed to use the known relation of the camera positions relative to the lighting as described above in order to calculate (predict) the variation of light intensity across the surface of the object. The system then matches those calculated smoothly-varying brightnesses with the measured brightnesses as detected by the cameras in the actual image. It is also contemplated that the system can be programmed to find the center of the specular reflection on the scan body and calculate where the center of the sphere would be given the relationship between the camera, light source and scan body based upon that reflection center.

The system next stores the data on the location and orientation of the implant fixture 20 into memory and adds or overlays that onto the previously stored image of the patient's mouth for use by medical personnel in the creation of the abutment(s) and crowns.

The present invention eliminates the need to use an inter-oral scanner to locate the scan bodies. Instead, the system can be used with an existing image tracking system, such as the X-Guide® Navigation System available from X-Nav Technologies, LLC, Lansdale, PA.

The method of operation of the system in the present invention is as follows. In one embodiment, the image guidance system 40 includes a plurality of cameras 42, preferably at least two, with lighting to facilitate capture of the images. The cameras 42 are located outside the oral cavity to capture images of the scan bodies 12 attached to the patient's teeth as shown in FIG. 3. A tracking component 44, such as described in U.S. Pat. Nos. 9,402,691 and 9,943,374, may also be attached to the patient to facilitate the detection and tracking of movement of the patient's mouth, and/or a surgical instrument or tool. A processing system 50 receives and processes the images captured by the cameras 42 to recognize the scan bodies 12 and triangulates the locations and orientations relative to each camera 42. The processing system 50 uses a reference dataset which defines a reference coordinate system based on alignment to a portion of the oral anatomy. The processing system 50 determines the location and orientation of the scan bodies 12 based on the reference dataset.

After the surgeon has added the implant fixture 20 into the patient's jawbone 22, the scan body 12 is threaded into the fixture. The system 40 is then activated so that the cameras 42 can capture the images of the scan body 12 in the patient's mouth. The processing system, such as the processor described above, determines the location and orientation of the scan body 12 and, from that, stores the location of the implant fixture. Then the processing system, using the transformations, converts the location of the implant fixture location and orientation to the appropriate coordinate system applicable to a previously captured CT scan of the patient's mouth. This process accurately determines the relationship between multiple scan bodies (and by extension, implants) so that an appliance that spans multiple implants will secure to the implants properly once manufactured. The system is determining the relationship of the scan bodies to the anatomy. The system is also configured to permit the overlay of the scan bodies location and geometry on top of a 3D surface derived from the CT scan.

The system or systems described herein may be implemented on any form of computer or computers and the algorithms and programs may be implemented as dedicated applications or in client-server architectures, including a web-based architecture, and can include functional programs, codes, and code segments. The computer system of the present invention may include a software program be stored on a computer and/or storage device (e.g., mediums), and/or may be executed through a network. The computer steps may be implemented through program code or program modules stored on a storage medium.

The processor 50 may include one or more processors for executing instructions. An example of processor 50 may include, but is not limited to, any suitable processor specially programmed as described herein, including a controller, microcontroller, application specific integrated circuit (ASIC), field programmable gate array (FPGA), system on chip (SoC), or other programmable logic or state machine. The processor 50 may include other processing components, such as an arithmetic logic unit (ALU), registers, and a control unit. The processor 50 may include multiple cores and may be able to process different sets of instructions and/or data concurrently using the multiple cores to execute multiple threads, for example.

A memory 52 may be configured for storing data (such as image data, models, location data) and/or computer-executable instructions defining and/or associated with the processor 50 for carrying out some or all of the processing or system steps described herein, and the processor 50 may retrieve and execute those instructions as contemplated herein. The memory 52 may represent one or more hardware memory devices accessible by processor 50. An example of memory 52 can include, but is not limited to, a type of memory usable by a computer, such as random access memory (RAM), read only memory (ROM), tapes, magnetic discs, optical discs, volatile memory, non-volatile memory, and any combination thereof. The memory 52 may include a main memory, preferably random access memory (RAM), and may also include a secondary memory. The secondary memory may include, for example, a hard disk drive and/or a removable storage drive, such as a floppy disk drive, a magnetic tape drive, an optical disk drive, flash drive, cloud storage, etc., and includes a computer usable storage medium having stored therein computer software and/or data.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The computer processes herein may be described in terms of various processing steps. Such processing steps may be realized by any number of hardware and/or software components that perform the specified functions. Aspects of the present disclosure may be implemented using hardware, software, or a combination thereof and may be implemented in one or more computer systems or other processing systems. In one aspect, the disclosure is directed toward one or more computer systems capable of carrying out the functionality described herein. For example, the described embodiments may employ various integrated circuit components, e.g., memory elements, processing elements, logic elements, look-up tables, and the like, which may carry out a variety of functions under the control of one or more microprocessors or other control devices. Similarly, where the elements of the described embodiments are implemented using software programming or software elements the invention may be implemented with any programming or scripting language such as C, C++, Java, assembler, or the like, with the various algorithms being implemented with any combination of data structures, objects, processes, routines or other programming elements. Functional aspects may be implemented in algorithms that execute on one or more processors. Furthermore, the embodiments of the invention could employ any number of conventional techniques for electronics configuration, signal processing and/or control, data processing and the like. The words “mechanism” and “element” are used broadly and are not limited to mechanical or physical embodiments, but can include software routines in conjunction with processors, etc.

The computer programs (also referred to as computer control logic, programming logic, or programming) are stored in the memory 52. Computer programs may also be received via a communications interface 54. Such computer programs, when executed, enable the computer system 50 to perform various features in accordance with aspects of the present disclosure, as discussed herein. In particular, the computer programs, when executed, enable the processor 50 to perform such features. Accordingly, such computer programs represent controllers of the computer system 50.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail.

Finally, the steps of all methods described herein are performable in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

1. A method of detecting a scan body mounted in the oral cavity of a patient, the method comprising the steps of:
receiving image data from each of a plurality of spaced apart cameras, the image data corresponding to images of a common location in a patient's oral cavity which includes one or more scan bodies, the image data including surface reflections from an outer surface of the one or more scan bodies from light emitted from at least one light source;
analyzing with a processor the image data to determine variations in the surface reflections of the light across the outer surface of at least one of the one or more scan bodies;
comparing the variations in the surface reflections to a reference dataset of prestored data representing reflectivity data associated with one or more scan bodies, the prestored data being associated with a reference coordinate system based on alignment to a portion of the patient's oral anatomy;
selecting from the reference dataset a subset of data representing reflectivity data that are closest to the variations in surface reflections in the image data;
retrieving a prestored three dimension model of the patient's oral cavity; and
depicting on the model the location and orientation of an implant fixture.

2. The method of detecting a scan body of claim 1, wherein the step of analyzing the image data comprises using the relation of each camera's position relative to the at least one light source.

3. The method of detecting a scan body of claim 1, wherein the step of analyzing the image data to determine variations in the surface reflections of the light comprises detecting a center of specular reflection of the surface reflection on a portion of one of the one or more scan bodies; and
the method further comprising the step of calculating where a center of a sphere would be based upon the center of the specular reflection, and locations of the cameras and the at least one light source.

4. The method of detecting a scan body of claim 3, wherein at least a portion of the scan body includes a curved surface defining a portion of sphere.

5. The method of detecting a scan body of claim 1 further comprising the step of tracking motion of a surgical tool relative to the implant location.

6. The method of detecting a scan body of claim 1, wherein prior to the step of depicting on the model the location and orientation of an implant fixture, the method involves the steps of receiving (i) stored values representing the camera's intrinsic characteristics, including focal length and distortion, (ii) location and extent for the at least one light source, and (iii) each scan body's reflectance and albedo properties; and determining a the pose of each scan body in the image; and determining a 3D rendering, location and orientation of the implant fixture.

7. The method of detecting a scan body of claim 6, wherein the 3D rendering is determined using Phong surface rendering/shading or ray-tracing.

8. The method of detecting a scan body of claim 1, wherein the step of analyzing the image data to determine variations in the surface reflections of the light comprises measuring the brightness as detected by the cameras in the image; and
wherein the step of comparing the variations in the surface reflections to a reference dataset involves using positions of the cameras relative to the at least one light source to calculate a variation of light intensity across a surface of at least one model scan body, and comparing the calculated variation of light intensity for the model scan body to the measured brightnesses of each scan body in the image to determine an orientation of the scan body in the image.

9. A method of detecting a scan body mounted in the oral cavity of a patient during optical imaging, the method comprising the steps of:
receiving image data from each of a plurality of spaced apart cameras, the image data corresponding to images of a common location in a patient's oral cavity which includes one or more scan bodies, the image data including surface reflections from an outer surface of the one or more scan bodies from light emitted from at least one light source;
analyzing with a processor the image data to determine variations in the surface reflections of the light across the outer surface of at least one of the one or more scan bodies, including using positions of the cameras relative to the at least one light source to calculate a variation of light intensity across a surface of at least one model scan body;
comparing the variations in the surface reflections to a reference dataset of stored data representing reflectivity data associated with one or more scan bodies, the stored data being associated with a reference coordinate system based on alignment to a portion of the patient's oral anatomy and determining an orientation of the scan body in the image based on the comparison;
determining a location of a surgical tool based on the orientation of the scan body; and
depicting on a display the location of the surgical tool.

10. The method of detecting a scan body of claim 9, wherein the step of analyzing the image data to determine variations in the surface reflections of the light comprises detecting a center of specular reflection of the surface reflection on a portion of one of the one or more scan bodies; and
the method further comprising the step of calculating where a center of a sphere would be based upon the center of the specular reflection, and locations of the cameras and the at least one light source.

11. The method of detecting a scan body of claim 10, wherein at least a portion of the scan body includes a curved surface defining a portion of sphere.

12. An optical imaging system for detecting and locating an implant fixture in the oral cavity of a patient using one or more scan bodies, the system comprising:
an imaging system comprising two or more spaced apart cameras, the cameras arranged to capture images of a common location;
a light source for illuminating the common location;
at least one memory including stored data representing (i) a reference dataset of reflectivity data from one or more scan bodies, the reference dataset being associated with a reference coordinate system based on alignment to a portion of the patient's oral anatomy, and (ii) a three dimension model of the patient's oral cavity from a prior scan of the patient's oral cavity;
a display for displaying data;
at least one processor programmed to perform the following:
receive image data from each of the plurality of spaced apart cameras, the image data including at least one scan body mounted in a patient's oral cavity;
analyze the image data to determine variations in light reflection from the light source across the outer surface of the at least one scan body to determine the reflectivity data for the images scan body;
retrieve the reference dataset;
compare the reflectivity data for the imaged scan body to reflectivity data for reference scan bodies in the reference dataset to select a subset of reference data representing reflectivity data for at least one reference scan body that is closest to the reflectivity data for the imaged scan body;
determine a pose and location of an implant fixture in the image data based on the comparison;
retrieve the prestored three dimension model of the patient's oral cavity; and
send data to the display representing the model and the pose and location of the implant fixture relative to the model.

13. The optical imaging system of claim 12 wherein the processor is programmed to compare the reflectivity data of the imaged scan body to the reflectivity data of the reference scan bodies in the reference dataset by modeling the appearance of the reflectivity data of the imaged scan body using known or detected lighting conditions and determining the alignment of the reference scan body that best describes the imaged scan body.

14. The optical imaging system of claim 12 wherein the reflectivity data is based on light reflectance characteristics of the outer surface of the scan body.

15. The optical imaging system of claim 14, wherein the light reflectance characteristics comprise differences in surface finish formed on the outer surface of the scan body at different locations.

16. The optical imaging system of claim 15, wherein the at least one memory includes stored data representing (i) a three dimensional location of the light source relative to an axis of each camera, and (ii) each camera's focal length and distortion characteristics, and wherein the reflectivity data for reference scan bodies includes the scan body's reflectance and albedo (reflective power) properties.

17. The optical imaging system of claim 16, wherein the step of determining a pose and location of an implant fixture involves creating a 3D rendering using a Phong surface rendering/shading process or a ray-tracing process.

18. The optical imaging system of claim 16, wherein the step of determining a pose and location of an implant fixture involves using an optimization algorithm involving (i) rendering a simulated image of a scan body, (ii) comparing the simulated image of the scan body with the actual imaged scan body and computing a visual similarity, and (iii) identifying a simulated image pose that generated the closest similarity score.

19. The optical imaging system of claim 14, wherein the step of analyzing the image data to determine variations in the surface reflections of the light comprises detecting a center of specular reflection of the surface reflection on a portion of one of the one or more scan bodies; and wherein the processor is programmed to calculate where a center of a sphere would be based upon the center of the specular reflection, and locations of the cameras and the at least one light source.

20. The optical imaging system of claim 19, wherein at least a portion of the scan body includes a curved surface defining a portion of sphere.
